(19) United States
(12) Patent Application Publication
(10) Pub. No.: US 2022/0073976 A1
(43) Pub. Date: Mar. 10, 2022

(54) CYCLONE ROLLING CIRCLE AMPLIFICATION, DOUBLE PRIMER FOR ROLONY AND REPLI-ROLONY AND PAIR-END SEQUENCING PROCESS

(71) Applicant: Qiagen Sciences, LLC, Germantown, MD (US)

(72) Inventors: Seiyu Hosono, Stoneham, MA (US); Michel Georges Perbost, Woburn, MA (US); Ryan Steven Hindman, Waltham, MA (US); Austin Ricker, Waltham, MA (US); Robert Pinard, Andover, MA (US); John Andrew Sheridan, Marblehead, MA (US)

(21) Appl. No.: 17/017,483

(22) Filed: Sep. 10, 2020

Related U.S. Application Data
(60) Provisional application No. 63/076,746, filed on Sep. 10, 2020.
(60) Provisional application No. 63/076,756, filed on Sep. 10, 2020.
(60) Provisional application No. 63/076,777, filed on Sep. 10, 2020.

(51) Int. Cl. C12Q 1/6855; C12N 15/10; C12Q 1/6848

(52) U.S. Cl. CPC C12Q 1/6855; C12Q 1/6848; C12N 15/1093

(57) ABSTRACT

Cyclone Rolling Circle Amplification (CRCA) based next-generation sequencing (NGS) using a multiple primer rolling circle amplification (RCA) reaction is generated using two or more tandem primers from the same strand on different locations of library adaptor regions of double stranded enriched targeted polymerase chain reaction (PCR) library products. This process allows multiple initiation and syntheses of the RCA reaction by an enzyme on the same circular template molecule, which is beneficial since the two or more primers complement each other in generating uniform amplification of the target circle population. Also, a method for keeping DNA nanoballs (also known as rolonies) compact, or more compact, and for pre-priming rolonies before sequencing to eliminate the hybridization of a seqeuncing primer after seeding the rolonies, and a Rolonies rolling circle amplification (RCA) based next-generation sequencing (NGS) using a dual Rolonies primer approach named REPLI-Rolony.

### PRIORITY CLAIM

This application claims priority to U.S. Provisional App. Ser. No. 63/076,746, filed Sep. 10, 2020, U.S. Provisional App. Ser. No. 63/076,756, filed Sep. 10, 2020, and U.S. Provisional App. Ser. No. 63/076,777, filed Sep. 10, 2020.

### BACKGROUND OF INVENTION

### Field of Invention

The present invention provides a next-generation sequencing method. More particularly, the present invention provides a cyclone rolling circle amplification based next-generation sequencing method, a method for keeping DNA nanoballs (also known as rolonies) compact, or more compact, and for pre-priming rolonies before sequencing to eliminate the hybridization of a seqeuncing primer after seeding the rolonies, and a Rolonies rolling circle amplification (RCA) based next-generation sequencing (NGS) using a dual Rolonies primer approach named REPLI-Rolony.

### Brief Description of Related Art

Conventional rolling circle amplification (RCA) is an isothermal nucleic acid amplification technique, which differs from polymerase chain reaction (PCR) techniques in that the polymerase continuously adds single nucleotides to a primer annealed to a circular template, which results in a long concatemer ssDNA that contains tens to hundreds of tandem repeats that are complementary to the circular template. Typically, a single RCA primer is used in the RCA technique.

### BRIEF SUMMARY OF THE INVENTION

The present invention provides a novel next-generation sequencing (NGS) method, which is referred to herein as cyclone rolling circle amplification (CRCA). CRCA is a multiple primer rolling circle amplification (RCA) reaction that is generated using two or more tandem primers from the same strand on different locations of library adaptor regions of double stranded enriched targeted PCR library products. This method allows multiple initiation and syntheses of the RCA reaction by an enzyme on the same circular template molecule, which is beneficial since the two or more primers complement each other in generating uniform amplification of the target circle population.

The present invention also provides a method for keeping DNA nanoballs (also known as rolonies) compact, or more compact, and for pre-priming rolonies before sequencing to eliminate the hybridization of a seqeuncing primer after seeding the rolonies, and a Rolonies rolling circle amplification (RCA) based next-generation sequencing (NGS) using a dual Rolonies primer approach named REPLI-Rolony.

The foregoing and other features of the invention are hereinafter more fully described below, the following description setting forth in detail certain illustrative embodiments of the invention, these being indicative, however, of but a few of the various ways in which the principles of the present invention may be employed.

### DETAILED DESCRIPTION OF THE INVENTION

All of the subject matter disclosed in U.S. Provisional App. Ser. No. 63/076,746, filed Sep. 10, 2020, U.S. Provisional App. Ser. No. 63/076,756, filed Sep. 10, 2020, and U.S. Provisional App. Ser. No. 63/076,777, filed Sep. 10, 2020, is hereby incorporated by reference as is fully rewritten herein.

CRCA is a multiple primer RCA reaction that uses two or more tandem primers from the same strand on different locations of library adaptor regions of double stranded enriched targeted PCR library products. The RCA reaction is initiated by the two (or more) primers simultaneously displacing each other and forming two intertwining common strands of RCA. The two intertwining common strands of clonally amplified RCA undergo folding and thus form RCA nanoballs, which can be seeded onto a Flowcell surface to be sequenced by Single Base Extension.

The present invention also provides a method for keeping DNA nanoballs (also known as rolonies) compact, or more compact, and for pre-priming rolonies before sequencing to eliminate the hybridization of a seqeuncing primer after seeding the rolonies. The method uses a dimerized sequencing primer to bind to the rolonies and keep two concatemers in proximity. The dimerized sequencing primer can be functional (e.g., to allow seqeuncing extensions) or not functional. And, a linker, which connects both primers, can be cleavable or not cleavable.

Finally, the present invention provides Rolonies rolling circle amplification (RCA) based next-generation sequencing (NGS) using a dual Rolonies primer approach named REPLI-Rolony. The Rolonies RCA reaction is performed using both Sense and Antisense primers. This allows the synthesis of both sense (+) and antisense (−) strand of DNA in a single Rolonies nanoball. The key to pair-end sequencing is so that both strand can be sequenced sequentially from the same Rolonies RCA nanoballs seed onto a single flow cell. The Rolony nanoballs are seeded onto the flow cell and NGS sequencing reaction is performed using two different primers sequentially, initially with a sequencing primer from sense strand (+) and performing 50-150 cycles followed by sequencing primer from anti-sense strand (−) and performing another 50-150 cycles. This allows sequencing from both (+) and (−) strand from the same rolony attached to the flow cell in the same position on a flow cell.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. A cyclone rolling circle amplification method that comprises generating a multiple primer rolling circle amplification reaction using two or more tandem primers from the same strand on different locations of library adaptor regions of double stranded enriched targeted polymerase chain reaction library products.

2. The method according to claim 1, wherein the rolling circle amplification reaction is initiated by the two or more tandem primers simultaneously displacing each other and forming two intertwining common strands of clonally amplified rolling circle amplification products that undergo folding and form nanoballs.

3. The method according to claim 2, further comprising collecting the nanoballs and sequencing the nanoballs by single base extraction.

4. A method for keeping rolonies compact or more compact, and for pre-priming rolonies before sequencing to eliminate hybridization of a seqeuncing primer after seeding the rolonies, comprising using a dimerized sequencing primer to bind to the rolonies and keep two concatemers in proximity.

5. The method according to claim 4, wherein the dimerized sequencing primer is functional.

6. The method according to claim 4, wherein the dimerized sequencing primer is not functional.

7. The method according to claim 4 wherein a linker connects both primers.

8. The method according to claim 7, wherein the linker is cleavable.

9. The method according to claim 7, wherein the linker is not cleavable.

10. A Rolonies rolling circle amplification based next-generation sequencing method comprising performing a Rolonies RCA reaction using both Sense and Antisense primers to synthesize both sense (+) and antisense (−) strands of DNA in single Rolonies nanoballs.

11. The method according to claim 10, wherein pair-end sequencing is performed so that both strands are sequenced sequentially in the same Rolonies RCA reaction, and wherein the single Rolonies nanoballs seed onto a single flow cell.

12. The method according to claim 11, further comprising performing a sequencing reaction on the single Rolonies nanoballs seeded onto the flow cell using two different primers sequentially, initially with a sequencing primer from sense strand (+) and followed by sequencing primer from anti-sense strand (−).
